(19) United States
(12) Patent Application Publication
(10) Pub. No.: US 2025/0099517 A1
(43) Pub. Date: Mar. 27, 2025

(54) Use of Lactobacillus reuteri Y7 strain for prevention or treatment of ocular diseases

(71) Applicants: Hui-Yu Huang, Taipei City (TW); Taipei Medical University, Taipei City (TW)

(72) Inventor: Hui-Yu Huang, Taipei City (TW)

(21) Appl. No.: 18/774,556

(22) Filed: Jul. 16, 2024

(30) Foreign Application Priority Data
Sep. 27, 2023 (TW) 112137103

(51) Int. Cl. A61K 35/747; A61P 27/02

(52) U.S. Cl. CPC A61K 35/747; A61P 27/02

(57) ABSTRACT

The probiotics, Lactobacillus reuteri Y7, are used in eye protection for preventing and treating ocular inflammatory reactions, apoptosis and retinal injuries after exposure to blue light. The exposure to blue light does harms to a retina such as blue light-induced retinal atrophy, retinal thickness thinned, macular choroidal neovascularization (CNV) and photosensitivity of a retina dysfunctional; the exposure to blue light causes retinal injuries such as the macular disease, particularly the late dry macular disease. Lactobacillus reuteri Y7 has the therapeutic effect on mammals like canines and felines in addition to human beings.

### REFERENCE TO RELATED APPLICATIONS

The present application is based on, and claims priority from, Taiwan application number 112137103, filed Sep. 27, 2023, the disclosure of which is hereby incorporated by reference herein in its entirety.

### REFERENCE TO AN ELECTRONIC SEQUENCE LISTING

The content of the electronic sequence listing (Substitute Sequence Listing.xml; Size: 27,571 bytes; and Date of Creation: Sep. 24, 2024) is herein incorporated by reference in its entirety.

### FIELD OF THE INVENTION

The present invention relates to a probiotic strain, Lactobacillus reuteri Y7, which is used in protecting eyes and preventing blue light-induced injuries to retinas.

### BACKGROUND OF THE INVENTION

The neurodegenerative diseases will replace cancers and become the second leading cause of death in the North America by 2040 according to epidemiological statistics. Neurodegeneration occurring imperceptibly and being known as the silent epidemic is common in elder patients who are gradually isolated from the society in the period of onset and ignored by the general public due to medical knowledge deficient currently. Following aging of the population structure, more and more patients diagnosed with neurodegenerative diseases deal a heavy blow to economics, the psychological level and lifestyles for families, patients and the public.

The age-related macular disease (AMD) is a critical ophthalmic disease that cause blindness in adults or the elderly. The World Health Organization (WHO) mentioned that AMD had become the primary cause of poor vision and contributed to a rising trend of visual deterioration or blindness. Despite 10% elder persons over 65 suffering from AMD in Taiwan in contrast to the higher percentage in the Occident, Taiwanese patients diagnosed with AMD might be short of the active treatment because of people's knowledge deficit for AMD and effective remedies. Accordingly, AMD has been one of primary causes of blindness in Taiwan and known as “number one killer of vision”.

In the early stage of AMD, the partial extracellular deposits, i.e., drusen, are found deposited at macula and the symptom of focal geographic chorioretinal thinning that affects the central or peripheral visual field is observed. When the progression of disease is choroidal neovascularization (CNV) in choroid membranes, the symptoms are subretinal fluid accumulation as well as localized-leak retinal detachment or subretinal hemorrhage.

In recent years, light injuries related to eyes and incidental topics have attracted more and more attentions. As one issue concerned by people for a long time, the incidence of ultraviolet injuries inflicted by sunlight, for example, sunburn and skin cancer, is still rising. Currently, the phenomena for widespread applications of light bulbs and digital devices emitting blue light (e.g., mobile phone and computer) have evoked our attention to blue light and people unanimously agreed that more researches and discussions are inevitable because a mammal's retinas are susceptible to oxidative stress. In fact, high-concentration reactive oxygen species (ROS) are easily generated by photoreceptors (PR) and retinal pigment epithelia (RPE) due to the following issues: (1) PR containing a variety of photosensitive molecules are frequently exposed to illumination; (2) The metabolism and functions of PR or RPE of a retina featuring high oxygen consumption work at the hyperoxic condition; (3) Photoreceptor discs rich in polyunsaturated fatty acids are vulnerable to oxidative stress and impaired photoreceptor discs are renewed through phagocytosis of RPE but cause a great deal of ROS accumulating on pigment epithelial cells. Theses processes bring risks to retinas, especially an elder person's oxidizable retinal neurons and RPE, deteriorating or damaging tissues.

The oxidative damages of retinas are usually attributed to eyes' prolonged contacts with photooxidation. Either a natural or an artificial light source probably leads to light/photic maculopathy and light-induced damages, if long-term accumulated, are considered as the primary cause of AMD, vision deterioration or blindness of an elder person. Scientists discovered the consistent pathological features were common in oxidative retinal injuries of test animals exposed to hard light as well as retinal injuries of human patients with late AMD. In fact, it has been demonstrated that the basic characteristics of human dry (non-neovascular) AMD were reproduced in the animal model for light-induced retinal injuries. As disclosed in the histological study, the features of AMD were presented in animals' retinas exposed to different levels of illumination, for example, apoptosis of PR, RPE transferred to neural retinas, interference in the blood-retinal barrier (BRB) and inflammatory infiltration. The animal model researches gave the general mechanism for researching basic retinal injuries of AMD and other important information of degenerative retinal diseases. Additionally, another relevant issue in the modern ophthalmology originates from the state-of-the-art ophthalmologic operations and ophthalmologic nursing instruments, both of which increase the incidence of heterologous photochemical damages. As disclosed in researches for albino rats, the morphology of retinas was negatively affected by brief exposure to blue light, for example, degeneration of pigmentary epithelium, deformation of the outer nuclear layer (ONL), PR loss, drusen, geographic atrophy (GA), CNV and others like terminal deoxynucleotidyl transferase (TdT) dUTP nick end labeling, apoptosis-inducing factors and necrosis-promoting receptor-interacting protein (RIP) kinases on retinal cells. Albino Wistar rats exposed to white/blue LED lamps (750 lux) for 28 days displayed more free radicals generated and the ONL thickness thinned. After albino Wistar rats and Long Evans rats were exposed to cool white, blue and green LED lamps (500-6000 lux) for 1 to 28 days, more PR losses and retinal toxicity in rats exposed to the blue component in cool white LED lamps were observed. In addition, high-brightness illumination promotes tight junction proteins of RPE such as Zonula-occludin 1 (ZO-1) deformed and destroyed and immediately affects BRB. In other reports, the molecular mechanism correlated with phototoxicity induced by blue light were published in succession. Accordingly, the long-term effect of blue light illumination on eyes due to widespread uses of smart phones needs to be further understood.

The probiotics such as Lactobacillus reuteri Y7 living in the digestive system, breast milk, skin and the urinary tract mostly can generate antibacterial molecules like organic acids, ethanol and reuterin. Particularly, reuteri with the antimicrobial activity inhibits colonization of pathogenic microorganism, remolds the composition of symbiotic microorganisms inside a host and contributes to the immune system of a host by moderating proinflammatory factors produced and promoting growth and functions of regulatory T cells; the colonization of L. reuteri for enhanced integrity of gut barriers alleviates microbes shifting to tissues from enteric cavities; the colonization of reuteri mitigates inflammatory diseases in intestines and distant tissues.

With 3C products popularizing, there have been more and more patients suffering from AMD. However, the methods to treat AMD are the laser therapy or the intraocular injection currently, which is declined by patients passively due to psychological fears.

Accordingly, how to offer edible probiotics for protecting eyes and effectively preventing or treating the macular disease inflicted by blue light is a critical issue disclosed in the present specification.

### SUMMARY OF THE INVENTION

The present invention discloses a probiotic strain named as Lactobacillus reuteri Y7, which was deposited under the number of BCRC911162 at the Food Industry Research and Development Institute in Taiwan. Lactobacillus reuteri Y7 was also internationally deposited with a deposit number of NITE BP-04078 under the Budapest Treaty at the National Institute of Technology and Evaluation, Patent Microorganisms Depository (NPMD) (Room No. 122, 2-5-8, Kazusakamatari, Kisarazu-shi, Chiba Prefecture, 292-0818), on Feb. 9, 2024.

The present invention also discloses a probiotic composition containing the probiotic strain of Lactobacillus reuteri Y7 with a therapeutically effective amount.

To this end, the probiotic composition is taken as a medicine, food or a food additive.

To this end, the Lactobacillus reuteri Y7 belongs to a viable bacterial strain.

To this end, the probiotic composition is characteristic of a formulation manufactured as a solution, a suspension liquid, an emulsion, powders, a pastille, a pill, syrup, an oral ingot, a tablet, a chewing gum, a thick juice, a colloid or a topical agent.

The present invention also discloses an application of Lactobacillus reuteri Y7 to be prepared as a pharmaceutical composition for eye protection.

To this end, the eye protection is aimed at preventing or treating ocular inflammatory reactions, apoptosis and retinal injuries of mammals after exposure to blue light.

To this end, the exposure to blue light does harms to a retina such as blue light-induced retinal atrophy, retinal thickness thinned, macular choroidal neovascularization (CNV) and photosensitivity of a retina dysfunctional.

To this end, the exposure to blue light causes retinal injuries such as macular disease.

To this end, the macular disease means the late dry macular disease.

To this end, the mammals can be human beings, pigs, canines, felines, bovines, horses, donkeys, deers, goats, sheeps, rabbits, mice, rats, guinea pigs or monkeys.

To this end, the blue light is characteristic of the wave length ranging from 380 nm to 530 nm.

To this end, the eye protection is aimed at maintaining integrity of gut barriers for alleviation of ocular inflammatory reactions, apoptosis and retinal injuries.

In summary, the new probiotic strain, Lactobacillus reuteri Y7, selected in the present disclosure proved effective in relieving the symptom of blue light-induced AMD through fundus photography (FP). As shown in SD-OCT and sections of tissues, both low-dosage Y7 probiotics and high-dosage Y7 probiotics maintained the retinal thickness effectively without retinal atrophy after long-term exposure to blue light. As shown in ERG, the intervention of low-dosage Y7 probiotics or particularly high-dosage Y7 probiotics maintained the a-wave (b-wave or c-wave) significantly, that is, Y7 probiotics effectively prevented a retina from blue light-induced photosensitivity dysfunctional.

Furthermore, the activity of antioxidant enzymes was enhanced through intervention of Y7 probiotics, as shown in ELISA; both ocular inflammatory reactions and apoptosis were relieved with Y7 probiotics, as shown in q-PCR; both tight junctions of a colon and integrity of gut barriers were maintained after intervention of Y7 probiotics.

Thus, Y7 probiotics have effects on preventing and treating AMD by enhancing the activity of antioxidant enzymes inside a body, relieving ocular inflammatory reactions and protecting a retina from blue light-induced structural injuries and photosensitivity dysfunctional.

### BRIEF DESCRIPTION OF THE DRAWINGS

The techniques of present invention would be more understandable from the detailed description given herein below and the accompanying figures are provided for better illustration, and thus description and figures are not limitative for present invention, and wherein:

FIG. 1 is an experimental flow chart of the present invention.

FIG. 2 shows experimental results for intraocular pressures wherein: NC: control group; BL: blue light group; LU: blue light+lutein group; Y7L: low-dosage Y7 probiotics group; Y7H: high-dosage Y7 probiotics group; #: p<0.05 for comparison with the NC group at the same time; *: p<0.05 for comparison with the BL group at the same time; data were analyzed with the one-way ANOVA and the Newman-Keuls test for the post hoc test; p<0.05 denotes a statistical difference.

FIG. 3a shows fundus photography (FP), fundus fluorescein angiography (FFA) and spectral-domain optical coherence tomography (SD-OCT) for fundi, retina status and distributions of fundus vessels of mice after intervention of blue light on DAY35; FIG. 3b shows results of the quantitative OCT analysis for all layers of a retina; data were written as Mean±SEM; NC: control group; BL: blue light group; Y7L: low-dosage Y7 probiotics group; Y7H: high-dosage Y7 probiotics group; LU: blue light+lutein group; n=6 in each group; *: significant difference relative to the NC group (p<0.05); #: significant difference relative to the BL group (p<0.05).

FIG. 4 shows electroretinography (ERG) for mice after intervention of blue light on DAY35; data were written as Mean±SEM; NC: control group; BL: blue light group; LU: blue light+lutein group; Y7L: low-dosage Y7 probiotics group; Y7H: high-dosage Y7 probiotics group; *: p<0.05 for comparison with the NC group at the same time; #: p<0.05 for comparison with the BL group at the same time; $: p<0.05 for comparison with the LU group at the same time; data were analyzed with the one-way ANOVA and the Newman-Keuls test for the post hoc test; p<0.05 denotes a statistical difference.

FIG. 5a shows fundus photography (FP), fundus fluorescein angiography (FFA) and spectral-domain optical coherence tomography (SD-OCT) for fundi, retinas and distributions of fundus vessels of mice before intervention of blue light on DAY0; FIG. 5b shows results of the quantitative OCT analysis for all layers of a retina; no lesion like atrophy was detected in any group; data were written as Mean±SEM; NC: control group; BL: blue light group; Y7L: low-dosage Y7 probiotics group; Y7H: high-dosage Y7 probiotics group; LU: blue light+lutein group; n=6 in each group.

FIG. 6a shows sections of retinal tissues stained by H&E wherein a fraction of retina on one section collected from a sacrificed mouse and stained by H&E was the central part below the pupil vertically and nearest to optic nerves; FIG. 6b shows retinal thicknesses analyzed with ImageJ; data were written as Mean±SEM; NC: control group; BL: blue light group; LU: blue light+lutein group; Y7L: low-dosage Y7 probiotics group; Y7H: high-dosage Y7 probiotics group; *: p<0.05 for comparison with the NC group at the same time; #: p<0.05 for comparison with the BL group at the same time; $: p<0.05 for comparison with the LU group at the same time; data were analyzed with the one-way ANOVA and the Newman-Keuls test for the post hoc test; p<0.05 denotes a statistical difference

FIG. 7a shows the activity of antioxidant enzyme, glutathione peroxidase (GPX), wherein 1 unit (U) is defined as the amount of enzyme that oxidizes 1.0 μmol NADPH to NADP+ per minute at 25° C.; FIG. 7b shows the activity of antioxidant enzyme, catalase (CAT), wherein 1 unit (U) is defined as the amount of CAT for dissolution of 1 μmol H₂O₂ per minute at 37° C. in 1 mL serum or plasma; data were written as Mean±SEM; NC: control group; BL: blue light group; LU: blue light+lutein group; Y7L: low-dosage Y7 probiotics group; Y7H: high-dosage Y7 probiotics; *: p<0.05 for comparison with the NC group at the same time; #: p<0.05 for comparison with the BL group at the same time; $: p<0.05 for comparison with the LU group at the same time; data were analyzed with the one-way ANOVA and the Newman-Keuls test for the post hoc test; p<0.05 denotes a statistical difference.

FIG. 8a shows inflammation-related gene expression such as MCP-1 through q-PCR based on extractions of RNAs from test animals' eyes; FIG. 8b shows inflammation-related gene expression such as IL-18 through q-PCR based on extractions of RNAs from test animals' eyes; FIG. 8c shows inflammation-related gene expression such as NLRP3 through q-PCR based on extractions of RNAs from test animals' eyes; FIG. 8d shows inflammation-related gene expression such as NF-KB p65 through q-PCR based on extractions of RNAs from test animals' eyes; FIG. 8e shows inflammation-related gene expression such as Caspase1 through q-PCR based on extractions of RNAs from test animals' eyes; FIG. 8f shows inflammation-related gene expression such as TNF-α through q-PCR based on extractions of RNAs from test animals' eyes; FIG. 8g shows inflammation-related gene expression such as IL-6 through q-PCR based on extractions of RNAs from test animals' eyes; FIG. 8h shows inflammation-related gene expression such as IL-1β through q-PCR based on extractions of RNAs from test animals' eyes; data were written as Mean±SEM; NC: control group; BL: blue light group; LU: blue light+lutein group; Y7L: low-dosage Y7 probiotics group; Y7H: high-dosage Y7 probiotics; *: p<0.05 for comparison with the NC group at the same time; #: p<0.05 for comparison with the BL group at the same time; $: p<0.05 for comparison with the LU group at the same time; data were analyzed with the one-way ANOVA and the Newman-Keuls test for the post hoc test; p<0.05 denotes a statistical difference.

FIG. 9a shows apoptosis-related gene expression such as Bax through q-PCR based on extractions of RNAs from test animals' eyes; FIG. 9b shows apoptosis-related gene expression such as Bcl-2 through q-PCR based on extractions of RNAs from test animals' eyes; FIG. 9c shows apoptosis-related gene expression such as Caspase3 through q-PCR based on extractions of RNAs from test animals' eyes; data were written as Mean±SEM; NC: control group; BL: blue light group; LU: blue light+lutein group; Y7L: low-dosage Y7 probiotics group; Y7H: high-dosage Y7 probiotics; *: p<0.05 for comparison with the NC group at the same time; #: p<0.05 for comparison with the BL group at the same time; $: p<0.05 for comparison with the LU group at the same time; data were analyzed with the one-way ANOVA and the Newman-Keuls test for the post hoc test; p<0.05 denotes a statistical difference.

FIG. 10a shows inflammation-related gene expression such as Occludin through q-PCR based on extractions of RNAs from test animals' eyes; FIG. 10b shows inflammation-related gene expression such as Cludin-1 through q-PCR based on extractions of RNAs from test animals' eyes; FIG. 10c shows inflammation-related gene expression such as ZO-1 through q-PCR based on extractions of RNAs from test animals' eyes; FIG. 10d shows inflammation-related gene expression such as Occludin through q-PCR based on extractions of RNAs from test animals' buccal mucosas; FIG. 10e shows inflammation-related gene expression such as Cludin-1 through q-PCR based on extractions of RNAs from test animals' buccal mucosas; FIG. 10f shows inflammation-related gene expression such as ZO-1 through q-PCR based on extractions of RNAs from test animals' buccal mucosas; FIG. 10g shows inflammation-related gene expression such as Occludin through q-PCR based on extractions of RNAs from test animals' colons;

FIG. 10h shows inflammation-related gene expression such as Cludin-1 through q-PCR based on extractions of RNAs from test animals' colons; FIG. 10i shows inflammation-related gene expression such as ZO-1 through q-PCR based on extractions of RNAs from test animals' colons; data were written as Mean±SEM; NC: control group; BL: blue light group; LU: blue light+lutein group; Y7L: low-dosage Y7 probiotics group; Y7H: high-dosage Y7 probiotics; *: p<0.05 for comparison with the NC group at the same time; #: p<0.05 for comparison with the BL group at the same time; $: p<0.05 for comparison with the LU group at the same time; data were analyzed with the one-way ANOVA and the Newman-Keuls test for the post hoc test; p<0.05 denotes a statistical difference.

### DETAILED DESCRIPTION OF THE INVENTION

The technical and scientific terminologies in the patent specification are well known to persons with common knowledge in the art unless specified otherwise; the materials mentioned in the patent specification are substances commercially available unless specified otherwise.

### Terminology Definition

The definitions of terminologies mentioned in the present specification and/or claims are introduced hereinafter. The definitions should be used to determine the meaning of the respective expressions unless the context requires a different meaning.

The terms ‘a’ or ‘an’ do not exclude a plurality, i.e., the singular forms ‘a’, ‘an’ and ‘the’ should be understood as to include plural referents unless the context clearly indicates or requires otherwise. In other words, all references to singular characteristics or limitations of the present disclosure shall include the corresponding plural characteristic or limitation, and vice versa, unless explicitly specified otherwise or clearly implied to the contrary by the context in which the reference is made. Therefor, the terms ‘a’, ‘an’ and “the” have the same meaning as ‘at least one’ or as ‘one or more’ unless defined otherwise. For example, reference to ‘an ingredient’ includes mixtures of ingredients, and the like.

The terms ‘about’ or ‘ca.’ will compensate for variability allowed in the pharmaceutical industry and inherent in pharmaceutical products, such as differences in content due to manufacturing variation and/or time-induced product degradation. The term allows for any variation in a real pharmaceutical, which would enable an evaluated product to be considered bioequivalent in a subject to enumerated benefits of a claimed product.

The term ‘composition’ means any type of composition in which multiple specific components as well as any other selected component are incorporated.

The terms ‘comprise’, ‘contain’ and similar expressions are to be construed in an open and inclusive sense, as ‘including, but not limited to’.

The terms ‘one embodiment’, ‘an embodiment’, ‘a specific embodiment’ and the like mean that a particular feature, property or characteristic, or a particular group or combination of features, properties or characteristics, as referred to in combination with the respective expression, is present in at least one of the embodiments of the invention. The occurrence of these expressions in various places throughout this description do not necessarily refer to the same embodiment. Moreover, the particular features, properties or characteristics may be combined in any suitable manner in one or more embodiments.

The term ‘pharmaceutically acceptable’ refers to a compound or mixture that is used in preparing a pharmaceutical composition, which is generally safe, non-toxic and neither biologically nor otherwise undesirable, and includes a compound or mixture acceptable for human pharmaceutical use.

The term ‘prevention’ refers to prevention of a disease, condition or symptom, as well as prevention of further growth and spread of progression or relapse after an initial improvement or after initial removal of the cause of the disease, condition or symptom.

The term ‘treatment’ refers to a therapeutic intervention for curing a disease, condition or symptom effectively; but ‘treatment’ also refers to improvement, amelioration, control, control of progression, prevention of progression, prevention of relapse, and the like.

The term ‘subject’ typically refers to humans. However, the invention is not limited to humans only and probably applicable to mammals if required. The term ‘mammals’ refers to animals classified as mammals and including, without limitation, human beings, livestock, farm animals, zoo animals, rodeo animals or pets, for example, human beings, primates, rodents, pigs, canines, felines, bovines, horses, donkeys, deers, goats, sheeps, rabbits, mice, rats, guinea pigs or monkeys.

The term ‘probiotics’ refers to viable and reproducible microorganisms and are well-known healthy microorganisms for a host, if administered properly, according to the technical level for application of the patent.

The term ‘therapeutically effective amount’ refers to the amount, which is sufficient to effectuate such treatment or prevention for a disease when administered to a subject for treating or preventing the disease.

### [Test Animals]

In the present invention, six-week-old male C57BL/6 mice were placed in a breeding cage in which the following feeding conditions were kept: temperature, 22±2° C., humidity, 55±5%, and the light-dark cycle of 12-hour light and 12-hour dark. The mice were fed with fodders at the adaptive phase for one week; general solid fodders for test animals were administered to mice of the control group and mice during the adaptive phase.

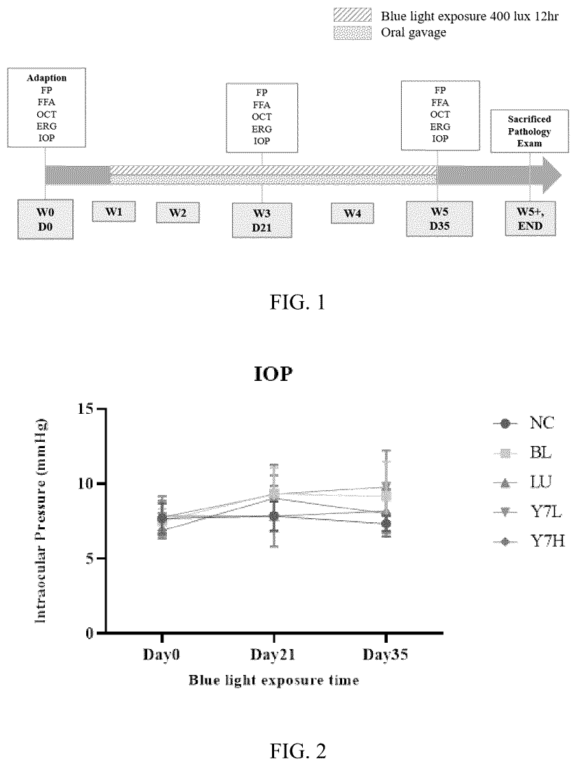

The initial day was defined as Day 0 when the adaptive phase for the mice was over. From the next week, the mice were interfered with samples under test and exposed to blue light. During the experiments, the mice were monitored for weights as well as water intakes and checked for any physiological change. The data for experiments through electroretinography (ERG), fndus photography (FP), fundus fluorescein angiograph (FFA), spectral-domain optical coherence tomography (SD-OCT) and intraocular pressure (IOP) were collected at Day 0, Day 21 and Day 35, as shown in FIG. 1 for the experimental process.

The C57BL/6 mice, which weighed between 23 and 25 g, were divided into seven groups randomly. In each group, the six mice induced by blue light successfully were incorporated in experiments. The samples dissolved in deuterium-depleted water (DDW) were fed to mice through oral tubes once a day. The groups were named and introduced as follows:
(1) Solvent not classified group (NC): The mice were fed with 0.2 ml deuterium-depleted water (DDW) through tubes;
(2) Negative control group (Blue light (BL)): The mice were fed with 0.2 ml deuterium-depleted water (DDW) through tubes and exposed to blue light;
(3) Low-dosage Y7 probiotics group (Y7L): The mice were fed with low-dosage Y7 probiotics (probiotic counts of 1.0×10⁸ CFU/ml dissolved in 0.2 ml DDW per day) and exposed to blue light;
(4) High-dosage Y7 probiotics group (Y7H): The mice were fed with high-dosage Y7 probiotics (probiotic counts of 1.0×10⁹ CFU/ml dissolved in 0.2 ml DDW per day) and exposed to blue light;
(5) Positive control group (lutein (LU)): The mice were fed with lutein and exposed to blue light. The recommended human dosage of lutein is 30 mg.

Referring to the human equivalent dose and the body surface area ratio, 12.3, for mouse (20 g) and human (60 kg), the daily dosage administered to a mouse is derived, that is, 30 mg/60 kg (human body weight)×12.3=6.15 mg/kg bw.

### Animal Model for Induction of the Macular Disease Through Blue Light Illumination

The mouse cage (48×25×20 cm) placed inside a stainless ventilated box was equipped with blue light boards as blue light sources around and above. In the experiment of animals exposed to blue light, six-week-old mice staying in the mouse cage were fed with drinking water as well as fodders and exposed to 400 Lux blue light illumination under the light-dark cycle of 12-hour light and 12-hour dark. The experiment was aimed at causing mice' late dry macular disease induced by chronic light injuries for simulating a human being's macular disease after long-term exposure to short-wavelength blue light from 3C products in daily life.

### Fundus Photography (FP) and Fundus Fluorescein Angiography (FFA)

Mice were anesthetized with Zoletil (40 mg/kg) and Xylazine (10 mg/kg) through intraperitoneal (IP). Atropine (0.125%) was distilled into eyes of a deeply anesthetized mouse placed on a clean bench for complete mydriasis after 30 minutes. Then, 2% Methocel gel as a corneal lubricant was distilled into the mouse's eyes for no injury attributed to excessive frictions between eyes and a fundus lens. Blood vessels of a fundus retina were checked with the Micron III retinal imaging microscope (Phoenix ResearY7H Laboratories); a mouse's central foveola was positioned at the center of the view window for capturing fundus photography (FP). Finally, the 10% sodium fluorescein solution was injected into a mouse through IP for FFA under a 520 nm lens.

### Spectral-Domain Optical Coherence Tomography (SD-OCT)

The OCT image system (Phoenix Research Laboratories, San Ramon, CA) used in the experiment is constructed based on the principle of spectral-domain optical coherence tomography. In the system, the computed tomography is divided into five layers, that is, Layer 1: the distance from the Ganglion cell layer (GY7L) to the outer plexiform layer (OPL); Layer 2: outer nuclear layer (ONL); Layer 3: the distance between the inner segment (IS) and the outer segment (OS); Layer 4: the distance from IS/OS to RPE; Layer 5: the total retinal thickness from the nerve fiber layer (NFL) to the lower edge of the RPE layer. The five layers were taken for the quantitative analysis by InSight XL (Phoenix Research Laboratories, San Ramon, CA).

### Electroretinography (ERG)

The mydriatic agent, Atropine (0.125%), was distilled into mice' eyes for mydriasis in advance. Then, mice were anesthetized with Zoletil (40 mg/kg) and Xylazine (10 mg/kg) through intraperitoneal (IP); 2% Methocel gel as a lubricant was distilled into eyes of a deeply anesthetized mouse having placed on a clean bench. The ERG analysis was enabled with the electrodes installed completely. A mouse was stimulated with single excitation light for collection of ERG from which a negative a-wave and a positive b-wave were generated. For the ERG analysis, the excitation time was defined as the period from the initial time of excitation light emitted to the time of the a-wave (b-wave) peak detected; the amplitude of a-wave was defined as the amplitude difference between the background ERG value at the initial time of excitation light emitted and the ERG value of the a-wave peak; the amplitude of b-wave was defined as the amplitude difference between the ERG value of the a-wave peak and the ERG value of the b-wave peak. Any change observed in the excitation time or an amplitude referred to functions of retinal cells normal or abnormal. In electrophysiology, the a-wave denotes the healthy condition of photoreceptors at outside of a retina; the b-wave denotes the healthy condition of medial nerve cells of a retina such as bipolar cells and horizontal cells; the c-wave denotes the healthy condition of retinal pigment epithelium (RPE) on a retina.

Real-Time Quantitative Polymerase Chain Reaction (q-PCR for Short)

The eyes of a mouse sacrificed were collected for the following purposes. RNAs extracted with Trizol were reverse-transcribed to cDNAs. Then, the real-time quantitative polymerase chain reaction was enabled to detect gene expressions related to inflammatory hormones and apoptosis such as TNF-α, IL-1B, IL-6, Bax, Bcl-2, Caspase1, Caspase3 and NLRP3 for comparisons with the endogenous level of β-actin, GAPDH or 18S rRNA. Primer sequences are shown in Table 1.

TABLE 1
gene sequence

| Gene | Forward | Reverse |
|---|---|---|
| NF-κB p65 | TCCTGTTCGAGTCTCCATGCAG (SEQ ID NO: 1) | GGTCTCATAGGTCCTTTTGCGC (SEQ ID NO: 2) |
| NLRP3 | TCACAACTCGCCCAAGGAGGAA (SEQ ID NO: 3) | AAGAGACCACGGCAGAAGCTAG (SEQ ID NO: 4) |
| MCP-1 | GCTACAAGAGGATCACCAGCAG (SEQ ID NO: 5) | GTCTGGACCCATTCCTTCTTGG (SEQ ID NO: 6) |
| IL-18 | GACAGCCTGTGTTCGAGGATATG (SEQ ID NO: 7) | TGTTCTTACAGGAGAGGGTAGAC (SEQ ID NO: 8) |
| TNF-α | GGTGCCTATGTCTCAGCCTCTT (SEQ ID NO: 9) | GCCATAGAACTGATGAGAGGGAG (SEQ ID NO: 10) |
| IL-1β | TGCCACCTTTTGACAGTGATG (SEQ ID NO: 11) | AAGGTCCACGGGAAAGACAC (SEQ ID NO: 12) |
| IL-6 | TACCACTTCACAAGTCGGAGG (SEQ ID NO: 13) | CTGCAAGTGCATCATCGTTGTTC (SEQ ID NO: 14) |
| Caspase1 | GGGCAAAGAGGAAGCAATTTATC (SEQ ID NO: 15) | GTGCCTTGTCCATAGCAGTAA (SEQ ID NO: 16) |
| Bax | TGCAGAGGATGATTGCTGAC (SEQ ID NO: 17) | CAAAGTAGAAGAGGGCAACCA (SEQ ID NO: 18) |
| Bcl-2 | AGAGCGTCAACAGGGAGAT (SEQ ID NO: 19) | GGGCCATATAGTTCCACAAAGG (SEQ ID NO: 20) |
| Caspase3 | GGAGTCTGACTGGAAAGCCGAA (SEQ ID NO: 21) | CTTCTGGCAAGCCATCTCCTCA (SEQ ID NO: 22) |
| Occludin | CTCCCATCCGAGTTTCAGGT (SEQ ID NO: 23) | GCTGTCGCCTAAGGAAAGAG (SEQ ID NO: 24) |
| Claudin-1 | GTTTGCAGAGACCCCATCAC (SEQ ID NO: 25) | AGAAGCCAGGATGAAACCCA (SEQ ID NO: 26) |
| ZO-1 | GCACCATGCCTAAAGCTGTC (SEQ ID NO: 27) | ACTCAACACACCACCATTGC (SEQ ID NO: 28) |
| GAPDH | GGAGCCAAACGGGTCATCATCTC (SEQ ID NO: 29) | GAGGGGCCATCCACAGTCTTCT (SEQ ID NO: 30) |

### Statistical Analysis

All data written as the form of mean±standard error of the mean (S.E.M.) were used in the statistical analysis through the one-way ANOVA and the Newman-Keuls test; p<0.05 denoted statistical difference. In the experiment, the software for the statistical analysis was Graph pad Prism (v9.2.0). The data such as food intake, water intake, body weight, organ weight and other clinical examination data were analyzed with the one-way ANOVA.

### Embodiment 1, Effects of Supplementation of Y7 Probiotics and Exposure to Blue Light on Mice' Physiologies

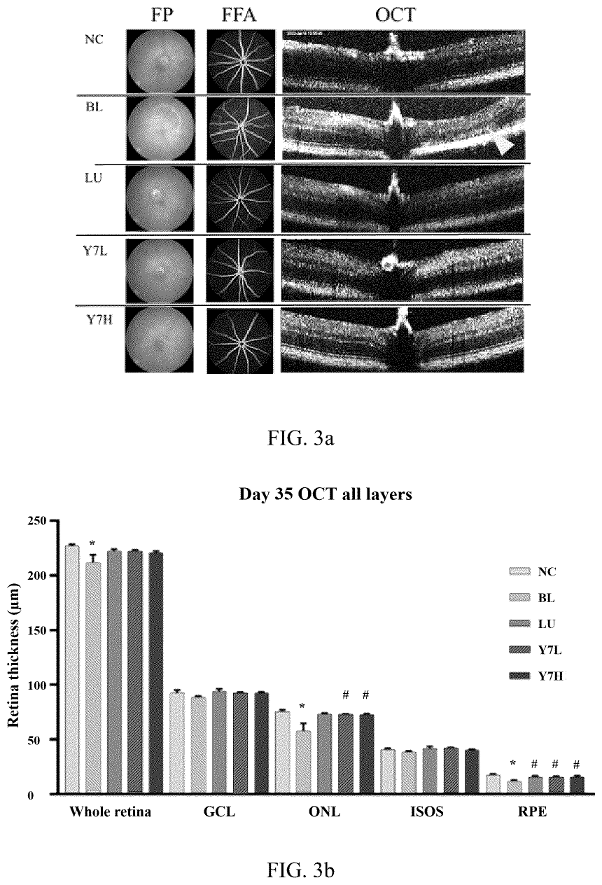

As shown in Table 2 for weights of mice fed with fodders and water during the experiment, the body weights of mice in the Y7H group were significantly higher than mice in the NC group at Days 7, 28 and 35. The food intakes of mice in the Y7H group were significantly higher than mice in the BL group, as shown in Table 3; there was no significant difference in water intakes of mice between groups, as shown in Table 4. According to IOP monitored by the iCare TA01i tonometer (ICAREFINLANDOY, Vantaa, Finland), the intraocular pressure of mice in the BL group showed an ascending trend in Week5 only. Accordingly, there was no high intraocular pressure after exposure to blue light during the experiment, as shown in FIG. 2. It can be seen from the experimental results that the animal model on the basis of blue light illumination had no effect on mice' physiologies in the experiment of Embodiment 1.

TABLE 2
body weights of mice during the experiment

| | Day 0 | Day 7 | Day 14 | Day 21 | Day 28 | Day 35 |
|---|---|---|---|---|---|---|
| NC | 21.35 ± 1.46 | 22.15 ± 1.79 | 22.07 ± 2.47 | 22.07 ± 2.47 | 22.80 ± 2.47 | 23.07 ± 2.24 |
| BL | 21.30 ± 1.67 | 21.90 ± 1.86 | 22.40 ± 1.89 | 22.40 ± 1.89 | 23.50 ± 2.55 | 23.97 ± 2.33 |
| LU | 21.25 ± 1.52 | 21.35 ± 1.97 | 22.12 ± 2.09 | 22.12 ± 2.09 | 22.40 ± 1.97 | 22.88 ± 2.86 |
| Y7L | 21.45 ± 0.59 | 22.47 ± 0.65 | 22.53 ± 0.57 | 22.53 ± 0.57 | 23.93 ± 0.29 | 24.20 ± 0.75 |
| Y7H | 22.67 ± 0.96 | 24.55 ± 1.68^{#}* | 24.60 ± 1.33 | 24.60 ± 1.33 | 26.13 ± 0.83* | 26.30 ± 1.04* |

The body weights of mice in the Y7H group were significantly higher than the NC group according to data recorded at Days 7, 28 and 35 during the experiment. All data were written as the form of mean±S.E.M.; NC: control group; BL: blue light group; LU: blue light+lutein group; Y7L: low-dosage Y7 probiotics group; Y7H: high-dosage Y7 probiotics group. *: p<0.05 for comparison with the NC group at the same time; #: p<0.05 for comparison with the BL group at the same time; $: p<0.05 for comparison with the LU group at the same time. Data were analyzed with the one-way ANOVA and the Newman-Keuls test for the post hoc test; p<0.05 denotes a statistical difference.

TABLE 3
food intakes of mice during the experiment

| | Day7 | Day14 | Day21 | Day28 | Day35 |
|---|---|---|---|---|---|
| NC | 5.46 ± 0.29 | 4.70 ± 0.56 | 4.59 ± 0.20 | 4.35 ± 0.64 | 4.47 ± 0.13 |
| BL | 5.74 ± 0.28 | 4.07 ± 0.78 | 4.32 ± 0.81 | 4.58 ± 0.95 | 4.20 ± 0.73 |
| LU | 5.71 ± 1.17 | 4.17 ± 1.15 | 5.04 ± 1.82 | 5.33 ± 2.17 | 4.43 ± 2.12 |
| Y7L | 4.16 ± 0.30 | 3.73 ± 0.20 | 4.56 ± 0.04 | 3.63 ± 0.46 | 4.54 ± 0.13 |
| Y7H | 4.46 ± 0.52 | 5.31 ± 0.77 | 5.61 ± 1.25 | 6.46 ± 1.47^{#} | 5.89 ± 0.40^{#} |

The food intake of mice in the Y7H group was significantly higher than the BL group at Day35. Data were written as Mean ± S.E.M.;
NC: control group;
BL: blue light group;
Y7L: low-dosage Y7 probiotics group;
Y7H: high-dosage Y7 probiotics group;
LU: blue light + lutein group;
n = 6 in each group.

TABLE 4
water intakes of mice during the experiment

| | Day7 | Day14 | Day21 | Day28 | Day35 |
|---|---|---|---|---|---|
| NC | 6.08 ± 2.93 | 7.11 ± 3.52 | 5.44 ± 0.69 | 4.73 ± 2.91 | 4.18 ± 0.56 |
| BL | 5.83 ± 3.69 | 4.96 ± 1.25 | 5.95 ± 1.13 | 4.32 ± 0.81 | 4.38 ± 0.88 |
| LU | 3.69 ± 0.86 | 4.17 ± 1.05 | 4.71 ± 0.42 | 3.65 ± 0.61 | 3.88 ± 0.31 |
| Y7L | 3.40 ± 0.06 | 4.77 ± 0.09 | 4.08 ± 0.13 | 3.50 ± 0.17 | 3.72 ± 0.14 |
| Y7H | 2.69 ± 0.19 | 3.05 ± 0.33 | 3.11 ± 0.27 | 3.04 ± 0.36 | 3.03 ± 0.69 |

There was no significantly difference in water intakes of mice between groups during the experiment. Data were written as Mean ± S.E.M.;
NC: control group;
BL: blue light group;
Y7L: low-dosage Y7 probiotics group;
Y7H: high-dosage Y7 probiotics group;
LU: blue light + lutein group;
n = 6 in each group.

### Embodiment 2, Validity of the Animal Model for the Blue Light-Induced Macular Disease on Mice

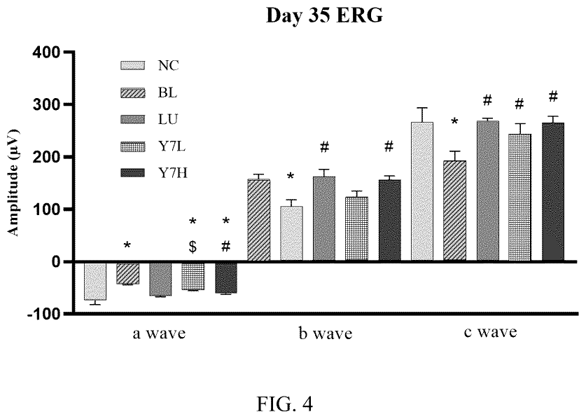

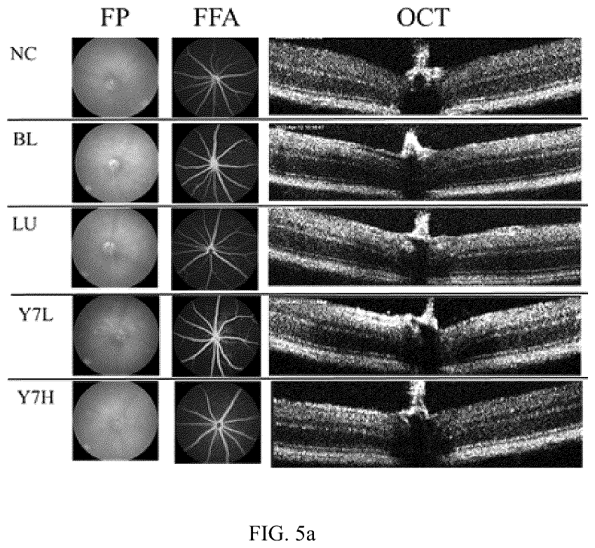

In the experiment, AMD was induced on mice successfully after five-week continuous intervention of blue light. As shown in FIG. 3a, the phenomena observed in mice of the BL group included: a larger deposit of drusen in the upper-right area of the fundus photography (FP); worse retinal atrophy at the right optic nerves according to SD-OCT; significantly thinned ONL, IS-OS and RPE layers which meant losses of PR and RPE cells; no symptom of choroidal neovascularisation (CNV) for late dry AMD according to FFA. As shown in FIG. 4, the a-wave (b-wave or c-wave) amplitude of the BL group was significantly lower than the control group or other intervention groups according to ERG. It means the activities of PR cells, bipolar cells, horizontal cells and RPE cells in a retina were impaired. In summary, the animal model for induction of late dry AMD with blue light was successful in Embodiment 2.

### Embodiment 3, Effects of Supplementation of Y7 Probiotics and Exposure to Blue Light on a Mouse' Fundus and Retina Structure

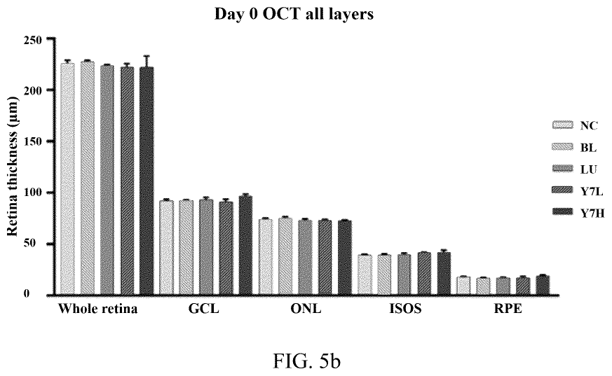

SD-OCT was used in checking lesions after exposure to blue light such as retinal atrophy and edema on a retina structure. According to the quantitative SD-OCT analysis, changes in all layers of a retina during the experiment were clearly inspected and no lesion like atrophy and edema was detected in any group before the experiment, as show in FIG. 5. Atrophy at the outer nuclear layer (ONL) of mice in the BL group was observed on Day 35, as shown by the arrow in FIG. 3a. With the quantitative SD-OCT analysis applied, more experimental results were observed: the total retinal thickness of mice in the BL group shrank significantly compared with the NC group (p<0.05); the ONL of mice in the BL group shrank significantly in contrast to the NC group (p<0.05); the significant difference was observed in ONLs between the Y7L group (the Y7H group) and the BL group (p<0.05); the RPE layer of mice in the BL group shrank significantly compared with the NC group (p<0.05); the fact that RPE layer of mice in the LU (Y7L or Y7H) group was significantly thicker than the BL group (p<0.05) implied blue light-induced retinal atrophy was relieved effectively due to intervention of Y7 probiotics, as shown in FIG. 3b.

As summarized in above experimental results, the total retinal thickness, the thickness of ONL or the thickness of the PRE layer shrank significantly when mice' eyes had been exposed to blue light. Because cell nuclei of PR lie in the ONL mostly, the losses of both PR cells receiving light as well as colors and PRE cells supplying nutrients to a retina and maintaining integrity of the retina structure will result in retinal atrophy. However, the thickness of a retina was effectively maintained by intervention of low-dosage Y7 probiotics, high-dosage Y7 probiotics or lutein for neither retinal atrophy nor eye functions impaired after long-term exposure to blue light. For that matter, the performance of the Y7H group was more similar to the control group than the LU group.

FFA was used in inspecting distributions of fundus vessels and determining the symptom of choroidal neovascularisation (CNV) for the late wet AMD. No CNV found in each group at Day 0 or Day 35 implied the late dry AMD was effectively controlled under the animal model for induction of the macular disease.

### Embodiment 4, Effect of Exposure to Blue Light on Photosensitivity of a Retina

In the experiment, the scotopic electroretinography (ERG) was used in inspecting influence of exposure to blue light on photosensitivity of a mouse's retina. When light was received by a mouse's eyes, opsins and 11-cis-retinal on photoreceptor cells were combined with each other for a visual cycle such that a membrane potential difference was produced for transmission of ERG by the retinal neuron layer sequentially, that is, the electroretinographic physiological function test was conducted through recording peak potential differences generated by different nerve cells. The experiment was aimed at making statistics of amplitude differences between negative a-waves from photoreceptor cells and positive b-waves from other retinal neurons for difference comparisons between groups. The a-wave denoted the activity of photoreceptor cells; the b-wave denoted the activity of bipolar cells or horizontal cells in a retina; the c-wave denoted the activity of RPE cells.

The experimental results were summarized in quantified amplitudes. The a-wave in ERG denoted the activity of photoreceptor cells. As shown by a-waves on Day 35 in FIG. 4, the amplitude of a-waves of the BL, Y7L or Y7H group decreased significantly compared with the NC group (p<0.05), the amplitude of a-waves of the Y7L group decreased significantly compared with the LU group (p<0.05), and the amplitude of a-waves of the Y7H group increased significantly compared with the BL group (p<0.05). These outcomes suggested the activity of PR in a retina was effectively maintained by high-dosage Y7 probiotics. The b-wave in ERG denoted the activity of bipolar cells or horizontal cells. As shown by b-waves on Day 35 in FIG. 4, the amplitude of b-waves of the BL group decreased significantly compared with the NC group (p<0.05) and the amplitude of b-waves of the BL group decreased significantly compared with the LU or Y7H group (p<0.05). These outcomes suggested the activity of bipolar cells or horizontal cells in a retina was effectively maintained by high-dosage Y7 probiotics. The c-wave in ERG denoted the activity of RPE. As shown by c-waves on Day 35 in FIG. 4, the amplitude of c-waves of the BL group decreased significantly compared with the NC group (p<0.05) and the significant difference was observed between the LU, Y7L or Y7H group and the BL group (p<0.05). These outcomes suggested the activity of RPE in a retina was effectively maintained after intervention of lutein or Y7 probiotics.

As summarized in the previous section for experimental results, the phenomena that the amplitude of a-waves, b-waves or c-waves of the BL group decreased significantly in contrast to the NC or intervention groups suggested impaired PR cells (bipolar cells, horizontal cells or RPE cells) contributed to photosensitivity degenerated. However, the amplitude of a-waves, b-waves or c-waves was maintained in the groups with intervention of Y7 probiotics, especially in the Y7H group with intervention of high-dosage probiotics which featured photosensitivity of a retina protected efficiently.

### Embodiment 5, Sections of Retinal Tissues after Supplementation of Y7 Probiotics and Exposure to Blue Light

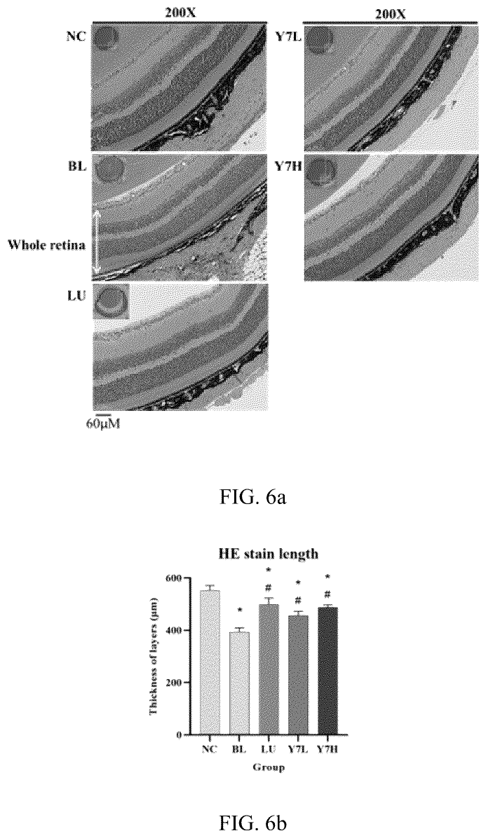

The eyes of a mouse sacrificed after the experiment on Day 35 were taken out for H&E stains. The section of a retinal tissue was checked for any change in each layer of a sacrificed mouse's retina and compared with experimental results of SD-OCT. The images for a fraction of retina below the pupil vertically and nearest to optic nerves were captured from a section through the 200× magnification, as shown in FIG. 6a. Then, the total retinal thickness was checked with ImageJ for the quantitative analysis, as shown in FIG. 6b. The total retinal thickness of mice in the BL, Y7L, Y7H or LU group decreased significantly compared with the NC group (p<0.05). However, the significant difference between the Y7L, Y7H or LU and the BL group (p<0.05) meant the retinal thickness was effectively maintained with intervention of Y7 probiotics or lutein, as shown in FIG. 6b, and particularly maintained based on dose-dependency of Y7 probiotics.

The above outcomes matched experimental results of SD-OCT in Week5, as shown in FIGS. 3a and 3b. Thus, the fact that Y7 probiotics relieved retinal injuries after exposure to blue light was validated.

### Embodiment 6, Analysis of Antioxidant Enzymes after Supplementation of Y7 Probiotics and Exposure to Blue Light

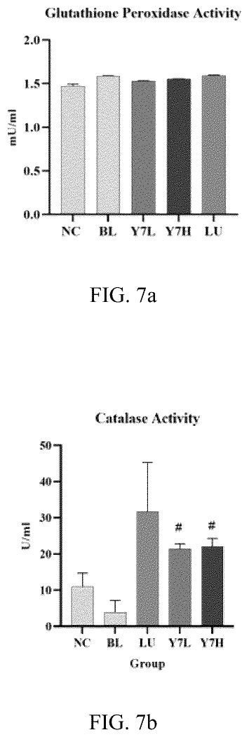

The performance of antioxidant enzymes in serums was analyzed through the enzyme-linked immunosorbent assay (ELISA). As shown in FIG. 7, there was no significant difference in the activity of glutathione peroxidase (GPX) between different groups. Comparatively, the activity of catalase (CAT) in the BL group showed a descending trend compared with the NC group but the activity of CAT in the Y7 groups (Y7H or Y7L) increased significantly compared with the BL group.

### Embodiment 7, Gene Expressions Related to Ocular Inflammation after Supplementation of Y7 Probiotics and Exposure to Blue Light

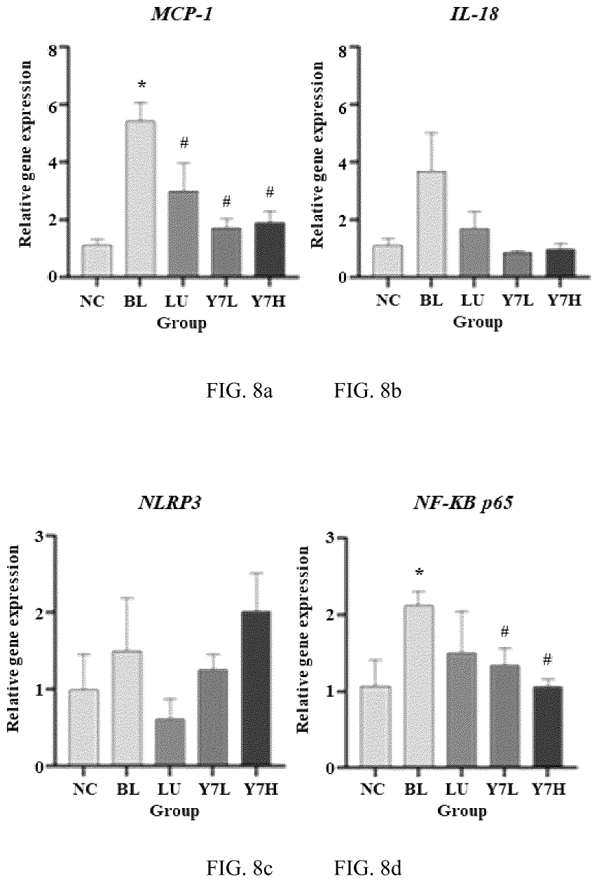

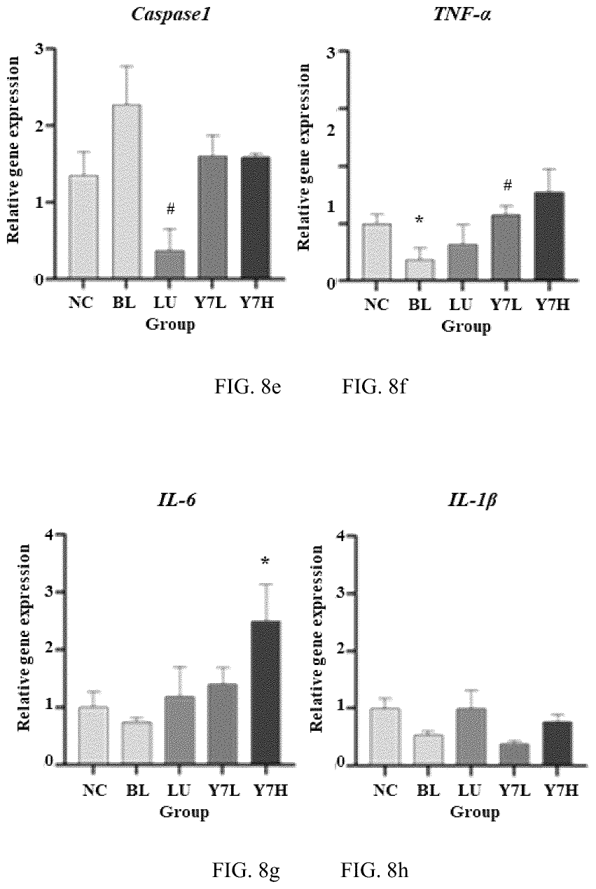

The gene expressions related to a mouse's ocular inflammation, for example, MCP-1 (FIG. 8a), IL-18 (FIG. 8b), NLRP3 (FIG. 8c), NF-KB p65 (FIG. 8d), Caspase1 (FIG. 8e), TNF-α (FIG. 8f), IL-6 (FIG. 8g) and IL-1β (FIG. 8h), were analyzed in the experiment. The gene expressions of both NF-KB p65 and MCP-1 in the BL group increased significantly compared with the NC group (p<0.05); the gene expressions of both NF-KB p65 and MCP-1 in the Y7L group or Y7H group reduced significantly compared with the BL group (p<0.05). Moreover, the inflammation-related gene expressions of NF-KB p65 and MCP-1 decreases as the dose of Y7 increases suggested the dose-dependent manner, as shown in FIGS. 8a and 8d. However, the statistically significant difference in NF-KB p65 was observed between the BL group and the NC group only and the statistic difference in NF-KB p65 was observed between the Y7 groups and the BL group, as shown in FIGS. 9a and 9b. The gene expressions of IL-18, NLRP3 or Caspase1 were increased in the BL group but decreased after intervention of Y7 probiotics in the Y7L and Y7H groups, as shown in FIGS. 8b, 8c and 8e, respectively. In summary, the intervention of Y7 probiotics relieved ocular inflammation attributed to blue light.

### Embodiment 8, Gene Expressions Related to Apoptosis in Eyes after Supplementation of Y7 Probiotics and Exposure to Blue Light

The gene expressions related to apoptosis in test mice' eyes, for example, Bax (FIG. 9a), Bcl-2 (FIG. 9b) and Caspase3 (FIG. 9c), were analyzed in the experiment. As shown in FIG. 9a, the gene expression of Bax in the BL group increased significantly compared with the NC group (p<0.05), the gene expression of Bax was significantly inhibited after intervention of Y7 probiotics and the gene expression of Bax in the Y7L group was closer to the NC group than the LU group; as shown in FIG. 9b, the gene expression of Bcl-2 in the Y7H group increased significantly compared with the BL group; as shown in FIG. 9c, the gene expression of Caspase3 in the BL group was more active than the NC group and the gene expression of Caspase3 was closer to the NC group after intervention of Y7 probiotics. In summary, apoptosis in mice' eyes attributed to blue light was effectively inhibited by Y7 probiotics.

### Embodiment 9, Gene Expressions Related to Tight Junctions of Tissues after Supplementation of Y7 Probiotics and Exposure to Blue Light

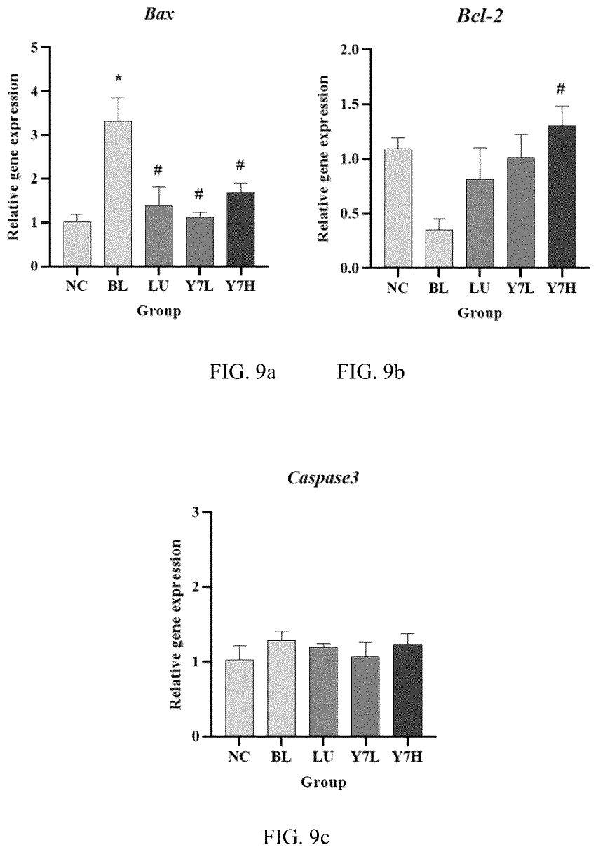

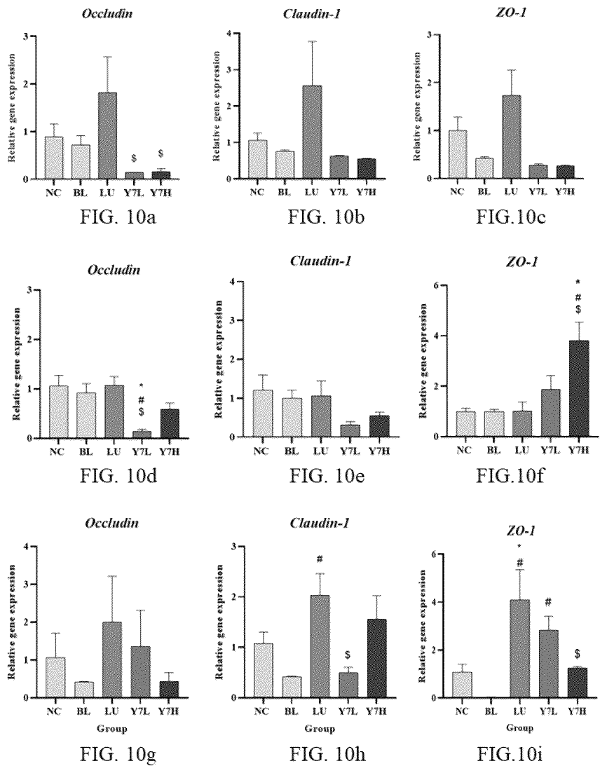

The gene expressions related to tight junctions of mice' eyes, buccal mucosas and colons after induction of blue light were analyzed through q-PCR and shown in FIG. 10a, FIG. 10d and FIG. 10g for Occludin, FIG. 10b, FIG. 10e and FIG. 10h for Claudin-1 and FIG. 10c, FIG. 10f and FIG. 10i for ZO-1, respectively. The gene expressions related to tight junctions of eyes in the BL group showed a descending trend compared with the NC group. The gene expressions of Occludin in the Y7L group and Y7H group were decreased significantly compared with the LU group (p<0.05) and the gene expressions of Claudin-1 or ZO-1 in each of Y7L and Y7H groups showed a descending trend compared with the LU group (p<0.05), as shown in FIG. 10a˜10c. The gene expressions related to tight junctions of buccal mucosas in the BL group showed a descending trend compared with the NC group. The gene expressions of ZO-1 in the Y7H group only were increased significantly compared with NC, BL or LU groups (p<0.05), as shown in FIG. 10d˜10f. The gene expressions related to tight junctions of colons in the BL group showed a descending trend compared with the NC group. The gene expressions of ZO-1 in both Y7L and Y7H groups were increased significantly (p<0.05) and the gene expressions of each of Occludin and Claudin-1 showed an ascending trend in both Y7L and Y7H groups, as shown in FIGS. 10g, 10 h and 10i.

Thus, Y7 probiotics were more effective in maintaining integrity of an intestinal tract than eyes or buccal mucosas.

In summary, the animal model for induction of the late dry AMD worked in the present invention successfully and was supported by evidences such as generation of drusen, obvious atrophies in the total retinal thickness, the ONL layer and the RPE layer, and photosensitivity dysfunctional; no CNV at a mouse's fundus after exposure to blue light was observed through FFA.

Y7 probiotics proved effective in relieving the symptom of blue light-induced AMD through fundus photography (FP). As shown in SD-OCT and sections of tissues, both low-dosage Y7 probiotics and high-dosage Y7 probiotics maintained the retinal thickness effectively without retinal atrophy after long-term exposure to blue light. As shown in ERG, the intervention of low-dosage Y7 probiotics or particularly high-dosage Y7 probiotics maintained the a-wave, b-wave or c-wave significantly, that is, Y7 probiotics effectively prevented a retina from blue light-induced photosensitivity dysfunctional.

Furthermore, the activity of antioxidant enzymes was enhanced through intervention of Y7 probiotics, as shown in ELISA; both ocular inflammatory reactions and apoptosis were relieved with Y7 probiotics, as shown in q-PCR; both tight junctions of a colon and integrity of gut barriers were maintained after intervention of Y7 probiotics.

Thus, Y7 probiotics have effects on preventing and treating AMD by enhancing the activity of antioxidant enzymes inside a body, relieving ocular inflammatory reactions and protecting a retina from blue light-induced structural injuries and photosensitivity dysfunctional.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

### [Biological Material Deposit]

Lactobacillus reuteri Y7 was deposited under the number of BCRC911162 at the Food Industry Research and Development Institute, Taiwan, on Nov. 29, 2022. Lactobacillus reuteri Y7 was also internationally deposited with a deposit number of NITE BP-04078 under the Budapest Treaty at the National Institute of Technology and Evaluation, Patent Microorganisms Depository (NPMD) (Room No. 122, 2-5-8, Kazusakamatari, Kisarazu-shi, Chiba Prefecture, 292-0818), on Feb. 9, 2024.

1. An application of Lactobacillus reuteri Y7 for preparation of a pharmaceutical composition used in eye protection, wherein the Lactobacillus reuteri Y7 is deposited at the NITE Patent Microorganisms Depository (NPMD) in Japan with a deposit number NITE BP-04078.

2. The application as claimed in claim 1, wherein the eye protection is aimed at preventing or treating ocular inflammatory reactions, apoptosis and retinal injuries of mammals after exposure to blue light.

3. The application as claimed in claim 2, wherein the exposure to blue light does harm to a retina such as blue light-induced retinal atrophy, retinal thickness thinned, macular choroidal neovascularization (CNV) and photosensitivity of a retina dysfunctional.

4. The application as claimed in claim 2, wherein the exposure to blue light causes retinal injuries, wherein the retinal injuries comprises macular disease.

5. The application as claimed in claim 4, wherein the macular disease is a late dry macular disease.

6. The application as claimed in claim 2, wherein the mammals comprise human beings, pigs, canines, felines, bovines, horses, donkeys, deer, goats, sheep, rabbits, mice, rats, guinea pigs or monkeys.

7. The application as claimed in claim 2, wherein the blue light has a wave length ranging from 380 nm to 530 nm.

8. The application as claimed in claim 2, wherein the eye protection comprises maintaining integrity of gut barriers for alleviation of ocular inflammatory reactions, apoptosis and retinal injuries.


SEQUENCE LISTING (30 sequences)

SEQ ID NO: 1 - DNA, 22 bp, synthetic construct
  source 1..22: mol_type = other DNA
tcctgttcgagtctccatgcag

SEQ ID NO: 2 - DNA, 22 bp, synthetic construct
  source 1..22: mol_type = other DNA
ggtctcataggtccttttgcgc

SEQ ID NO: 3 - DNA, 22 bp, synthetic construct
  source 1..22: mol_type = other DNA
tcacaactcgcccaaggaggaa

SEQ ID NO: 4 - DNA, 22 bp, synthetic construct
  source 1..22: mol_type = other DNA
aagagaccacggcagaagctag

SEQ ID NO: 5 - DNA, 22 bp, synthetic construct
  source 1..22: mol_type = other DNA
gctacaagaggatcaccagcag

SEQ ID NO: 6 - DNA, 22 bp, synthetic construct
  source 1..22: mol_type = other DNA
gtctggacccattccttcttgg

SEQ ID NO: 7 - DNA, 23 bp, synthetic construct
  source 1..23: mol_type = other DNA
gacagcctgtgttcgaggatatg

SEQ ID NO: 8 - DNA, 23 bp, synthetic construct
  source 1..23: mol_type = other DNA
tgttcttacaggagagggtagac

SEQ ID NO: 9 - DNA, 22 bp, synthetic construct
  source 1..22: mol_type = other DNA
ggtgcctatgtctcagcctctt

SEQ ID NO: 10 - DNA, 23 bp, synthetic construct
  source 1..23: mol_type = other DNA
gccatagaactgatgagagggag

SEQ ID NO: 11 - DNA, 21 bp, synthetic construct
  source 1..21: mol_type = other DNA
tgccaccttttgacagtgatg

SEQ ID NO: 12 - DNA, 20 bp, synthetic construct
  source 1..20: mol_type = other DNA
aaggtccacgggaaagacac

SEQ ID NO: 13 - DNA, 21 bp, synthetic construct
  source 1..21: mol_type = other DNA
taccacttcacaagtcggagg

SEQ ID NO: 14 - DNA, 23 bp, synthetic construct
  source 1..23: mol_type = other DNA
ctgcaagtgcatcatcgttgttc

SEQ ID NO: 15 - DNA, 23 bp, synthetic construct
  source 1..23: mol_type = other DNA
gggcaaagaggaagcaatttatc

SEQ ID NO: 16 - DNA, 21 bp, synthetic construct
  source 1..21: mol_type = other DNA
gtgccttgtccatagcagtaa

SEQ ID NO: 17 - DNA, 20 bp, synthetic construct
  source 1..20: mol_type = other DNA
tgcagaggatgattgctgac

SEQ ID NO: 18 - DNA, 21 bp, synthetic construct
  source 1..21: mol_type = other DNA
caaagtagaagagggcaacca

SEQ ID NO: 19 - DNA, 19 bp, synthetic construct
  source 1..19: mol_type = other DNA
agagcgtcaacagggagat

SEQ ID NO: 20 - DNA, 22 bp, synthetic construct
  source 1..22: mol_type = other DNA
gggccatatagttccacaaagg

SEQ ID NO: 21 - DNA, 22 bp, synthetic construct
  source 1..22: mol_type = other DNA
ggagtctgactggaaagccgaa

SEQ ID NO: 22 - DNA, 22 bp, synthetic construct
  source 1..22: mol_type = other DNA
cttctggcaagccatctcctca

SEQ ID NO: 23 - DNA, 20 bp, synthetic construct
  source 1..20: mol_type = other DNA
ctcccatccgagtttcaggt

SEQ ID NO: 24 - DNA, 20 bp, synthetic construct
  source 1..20: mol_type = other DNA
gctgtcgcctaaggaaagag

SEQ ID NO: 25 - DNA, 20 bp, synthetic construct
  source 1..20: mol_type = other DNA
gtttgcagagaccccatcac

SEQ ID NO: 26 - DNA, 20 bp, synthetic construct
  source 1..20: mol_type = other DNA
agaagccaggatgaaaccca

SEQ ID NO: 27 - DNA, 20 bp, synthetic construct
  source 1..20: mol_type = other DNA
gcaccatgcctaaagctgtc

SEQ ID NO: 28 - DNA, 20 bp, synthetic construct
  source 1..20: mol_type = other DNA
actcaacacaccaccattgc

SEQ ID NO: 29 - DNA, 23 bp, synthetic construct
  source 1..23: mol_type = other DNA
ggagccaaacgggtcatcatctc

SEQ ID NO: 30 - DNA, 22 bp, synthetic construct
  source 1..22: mol_type = other DNA
gaggggccatccacagtcttct